(19) United States
(12) Patent Application Publication
(10) Pub. No.: US 2022/0370536 A1
(43) Pub. Date: Nov. 24, 2022

(54) Preventing, treating, and curing illness by denaturing proteins and nucleic acids

(71) Applicant: Wallace Stuart McCloy, III, Memphis, TN (US)

(72) Inventor: Wallace Stuart McCloy, III, Memphis, TN (US)

(21) Appl. No.: 17/328,992

(22) Filed: May 24, 2021

(51) Int. Cl. A61K 36/534; A61K 36/53; A61K 31/047; A61K 31/19; A61K 31/17; A61K 33/00; A61K 47/10; A61K 47/12; A61K 47/46; A61P 31/00

(52) U.S. Cl. CPC A61K 36/534; A61K 36/53; A61K 31/047; A61K 31/19; A61K 31/17; A61K 33/00; A61K 47/10; A61K 47/12; A61K 47/46; A61P 31/00

(57) ABSTRACT

This disclosure describes how to prevent, treat, and cure illness through the use of denaturing agents.

### FIELD OF THE INVENTION

This invention spans many disciplines involved in life and touches parts of many fields including: biochemistry, botany, cell biology, dermatology, entomology, enzymology, herpetology, ichthyology, infectious disease, microbiology, molecular genetics, organic chemistry, parasitology, pathophysiology, physical chemistry, and virology.

### Background

This is novel technology which provides new solutions to old problems. This is not an obvious technology or else there would not be tens of thousands of people dying each year and billions of people suffering from diseases and illnesses globally which may have been prevented and cured if this technology had been utilized.

### BRIEF SUMMARY

This technology provides a method for preventing, treating, or curing a vertebrate patient's illness by administering a treatment regimen which involves steps including one or more steps wherein the proteins and nucleic acids of the patient as well as the proteins and nucleic acids of the infection, infectious agent, and illness-causing agent are denatured.

### DETAILED DESCRIPTION OF THE INVENTION

This invention is a powerful tool to help prevent morbidity and mortality in patients. However, this invention is not a panacea.

The claims section of this patent application provides information which is also crucial and essential to the understanding of the invention, and i reference it here so that readers may read the claims section of this patent application first to give understanding and context to which i will hereby add additional information and examples for application of this invention.

Humans are vertebrates, and the examples that follow will focus on human patients. Non-human vertebrates may also benefit from this invention.

Many illnesses that affect vertebrates are evident in the skin, and illnesses may manifest in the skin and even be transmitted by the skin. The skin is the largest organ of the human body. For these examples, we will administer this invention to prevent, treat, or cure a vertebrate patient's illness as a result of an infection, infectious agent, or illness-causing agent in or on the patient's skin.

Some reasons to use this invention may include, but are not limited to treat an infection, infectious agent, or illness-causing agent which is suspected to or believed to have infected the skin, is evident in the skin, is manifesting in the skin.

A virus is an obligate intracellular parasite composed of a protein coat and nucleic acid (DNA or RNA) with or without an envelope.

Viruses that infect the skin, or are evident in the skin, or manifest in the skin may be recommended for treatment by this invention.

A bacterium is a unicellular prokaryotic organism that has neither a true nucleus nor membrane-bounded organelles.

Bacteria that infect the skin, or are evident in the skin, or manifest in the skin may be recommended for treatment by this invention.

Toxins, venoms, and poisons made by animals, fish, insects, other creatures, or plants are largely made up of proteins or peptides (a peptide is a short protein), and these aforementioned toxins, venoms, and poisons (proteins or peptides) may be defeated by the denaturing effects of the effector formulations utilized by this invention.

Some reasons to use this invention may include but are not limited to treating an infection, infectious agent, or illness-causing agent due to any bacteria, parasite, plant toxin, virus, or any other causative factor included, or not specifically included, herein in the examples section.

Some reasons not to use this invention regarding bacteria and viruses may include but are not limited to treating an infection, infectious agent, or illness-causing agent which:
1. Does not transmit infection or illness to the skin.
2. Is proven to have, or is believed to have infected other organs, systems, tissues not of the skin such that the infection or illness cannot be treated in a localized fashion by a specific access point or area of the patient's skin.
3. Has already become, or caused the manifestation of, a systemic disease or illness in the specific patient such that the local effector formulation treatment regimen therapy will not benefit that particular patient because of the manifestation of that particular illness, infection, infectious agent, or illness-causing agent, and that particular disease process.
4. This invention is not designed to treat cancer.
5. This invention is not designed to treat warts.

Some reasons not to use this invention regarding plant toxins may include but are not limited to treating a plant toxin which:
1. Is not made up of proteins or peptides.

Plant toxins not made up of proteins or peptides are not recommended for treatment by this invention.

The spitting cobra spits venom at the eyes of vertebrates (as a protective mechanism wherein the snake defends itself by spitting venom), and the eyes of a patient would be a specific therapy location on a patient not to treat with this invention. However, if the spitting cobra bit a patient's arm, leg, torso, or other body part, then treatment with this invention of the patient's arm, leg, torso, or other body part (with the exclusion of the eyes) may be utilized.

This invention is not intended nor designed to be used in or on a patient's eye or eyes.

Skin is a large organ made up of many cells composing two main tissues: epithelial tissue and connective tissue. For the purpose of describing the invention and the application of the invention, let's define skin as all of the epithelial tissue cells and connective tissue cells inward toward the center of the body, body part, head, limb, torso, or appendage as appropriate from the outermost skin cell, as well as any other cell or tissue type native to that body which is inhabiting the same general location or area as the aforementioned skin, for example: basal lamina, collagen fibers, elastic fibers, fibroblasts, hair, keratinocytes, langerhans cells, mast cells, macrophages, melanocytes, lymphocytes, as well as other cell types in the same general location of the skin not specifically mentioned in this list, and in a precise location, which refers to the specific location of the patient's body that you can touch with your finger, an instrument, or tool. This invention and the technology associated with it may also be used to treat body parts, epithelial cells, mucosal surfaces, organs, skin, and tissues, whereby the surface treated may be located in the mouth, nose, oral cavity, urethra, vagina, and other locations whereby this definition of the skin regarding orientation may be reversed such that the direction will be outward, away from the center of the body, body part, head, limb, torso, or appendage, as appropriate, from the innermost skin cell. When describing a specific location on a patient's body or an illness or infection occurring at a specific location on a patient's body, then the specific location will be defined as the specific therapy location of the patient's body. For our example purposes, directions will be indicated as appropriate or as necessary, if necessary, depending upon what is believed by the inventor to be a good description.

Skin is made up of many cells, and the cells of vertebrates, including those making up human skin cells, have many membranes and membrane-bound compartments, or organelles, including: endoplasmic reticulum, endosome, golgi apparatus, lysosome, mitochondrion, nucleus, peroxisome, plasma membrane, and ribosome (not membrane-bound). The cytosol makes up more than 50% of many cells, and this cytosol is a water-based environment where lots of chemical reactions may take place. There are cell-to-cell channels, channels, connexons, gap junctions, and pores that serve as passageways to allow ions and small molecules (like the acid(s) or base(s) and other molecules of the effector formulation(s) example(s)) to readily pass from one side of a membrane to the other, and these features help the diffusion of the denaturing agents to act effectively, efficiently, and quickly. This invention uses effector formulations to cause a denaturing effect upon proteins and nucleic acids of the infection, infectious agent, and illness-causing agent as well as the proteins and nucleic acids of the cells of the patient's body wherein the infection, infectious agent, and illness-causing agent will not be able to survive, replicate, or further the disease process.

Human skin has been documented to have different native pH depending upon the location on the body and some difference between patients may also be expected. The body does work to keep native pH in an acceptable range through homeostasis.

Acids and bases have a strong denaturing effect on proteins and nucleic acids, so an effector formulation may comprise one or more ingredient(s) which may or may not include an acid or acids. An effector formulation may comprise one or more ingredient(s) which may or may not include a base or bases. Any effector formulation with a pH of less than 7 may be considered acidic, and any effector formulation with a pH of greater than 7 may be considered basic. An effector formulation is not limited to acids and bases. However, for these examples that follow in this detailed description of the invention section, we will consider an effector formulation which will include only two ingredients. Effector formulations may contain multiple ingredients not limited in any way except that the denaturation of proteins and nucleic acids must result after application for a certain period of time. The acidic effector formulation will include an acid ingredient plus water. The basic effector formulation will include a base ingredient plus water.

An effector solution does not have to contain any water or other solvent, but it may.

There may or may not be modulators added to the effector formulation which may increase the pH or decrease the pH, but these are not required.

Factors related to the denaturation process involve the strength of the cumulative pH of the effector formulation at the site where an infection, infectious agent, or illness-causing agent can come into close proximity with the effector formulation such that interaction may occur as well as the amount of time the effector formulation is directed to interact at that specific site with the applicable proteins and nucleic acids involved in the particular illness-causing episode.

If a strong acid, like an acid with a pH of 1.0, is applied topically to the skin, it will cause the pH of the skin to go down and become more acidic in the specific location where the acid is applied topically and also will cause the pH to become lower throughout the skin as well as into the skin deeper from the surface from the point of acid application. Likewise, if a strong base, like a base with a pH of 14.0, is applied topically to the skin, it will cause the pH of the skin to go up and become more basic in the specific location where the base is applied topically and also cause the pH to become higher throughout the skin as well as into the skin deeper from the point of base application. Effector formulations applied to the skin also have a strong tendency to move laterally in all directions away from the point of application at a specific therapy location.

Let's say this pH 1.0 acid is a liquid applied liberally to the surface of the patient's skin in a very specific location as big as the heads side of a $0.25, quarter, U.S. currency coin. Upon application, the acid will contact the skin and those plentiful acid molecules and hydrogen ions will start moving from the area of highest concentration to an area of lower concentration and they will do this quickly, effectively, and efficiently. Likewise, let's say that a pH 14.0 base is a liquid applied liberally to the surface of a different patient's skin in a very specific location as big as the heads side of a $0.25, quarter, U.S. currency coin. Upon application, the base will contact the skin and those plentiful base molecules with their respective hydroxide ions will start moving from the area of highest concentration to an area of lower concentration whereby they will readily accept hydrogen ions and they will also do this quickly, effectively, and efficiently. Effector formulations applied to the skin also have a strong tendency to move laterally in all directions away from the specific therapy location.

The effector formulation, the pH 1.0 acid, or the pH 14.0 base in these examples, will denature proteins and nucleic acids in the specific therapy location on a patient's body beginning upon application of the effector formulation, and continuing for some time after application with the effector formulation.

If there is an infection, infectious agent, or illness-causing agent present in the patient's body in the specific therapy location, then the effector formulation will cause changes to occur through localized chemical reactions in the patient's body to the infection, infectious agent, or illness-causing agent including those proteins and nucleic acids that are present in the infection, infectious agent, or illness-causing agent as well as to the patient's native proteins and the patient's native nucleic acids within the specific therapy location of the patient's body thereby potentially changing the infection's protein, infectious agent's protein, or illness-causing agent's protein as well as the patient's protein whereby the quaternary structure, tertiary structure, and secondary structure of the aforementioned protein which before the denaturing treatment had been in the native state and then after the denaturing effects of the effector treatment there is change to some or all of the aforementioned proteins in the specific therapy location of the patient's body which causes some or all of the proteins to do any one or more or all of the following: lose their shape, not fit properly, dissociate from sub-units, not function properly biologically, and the denaturing effect of the effector formulation as part of the treatment regimen may also result in the unfolding of polypeptides, disruption of intramolecular bonds which may include disulfide bridges, electrostatic, hydrophobic, non-covalent dipole-dipole interactions, and van der waals interactions, and potentially disrupts the alpha-helices and beta-pleated sheets such that they may also lose some or all of their respective native configurations and the denaturation effects upon these proteins may or may not be reversible and likewise the nucleic acids present in the same patient's body in the same specific therapy location as well as the infection's nucleic acids, infectious agent's nucleic acids, or illness-causing agent's nucleic acids within the specific therapy location which may or may not function properly biologically because of any one or more or all of the following: breaking of hydrogen bonds between watson and crick base pairs, losing their shape, potential unfolding of helices, such that the cumulative effect of the localized denaturation upon the infection, infectious agent, or illness-causing agent as well as the patient's own cells and including those patient proteins and nucleic acids within the patient's cells in the specific therapy location of the patient's body will be such that the infection, infectious agent, or illness-causing agent will not be able to survive, replicate, or further the disease process.

As soon as an indication to use the invention is realized, Prompt and thorough treatment with the invention is recommended.

Some of the examples of infection, infectious agent, or illness-causing agent must be treated immediately, and others may have a longer incubation period. Also, there are not always outward signs to indicate the presence of infection, infectious agent, or illness-causing agent in the skin.

Taking action by applying this invention on the skin in a timely fashion to the proper location is the key to defeating illness and the disease process, and is ultimately, the key to patient health.

Many times there is an event or episode with a creature before the problems start and the patient's health is negatively affected: I was bitten, scratched, or stung by a creature.

Sometimes it is an event or episode with a plant: i walked into the bush and then immediately i felt stinging and my skin hurt and then the skin began to swell.

Many times a patient has evidence, an outward sign to indicate the presence of infection, infectious agent, or illness-causing agent in the skin, that there is a health problem because you see evidence in the skin: there is evidence of a blister, bump, parasite, pustule, rash, sore, swelling, ulcer, or wound . . . it hurts, it itches, it looks bad, it looks different, or there is pain.

Some more things to consider regarding this invention technology include but are not limited to:
1. Time elapsed between the time of initial introduction of the infection, infectious agent, or illness-causing agent to the skin of the patient, by whatever means, and the time of application of the invention to the specific therapy location is very important.
2. Some instances wherein a creature injects a toxin deep into tissue, like with the fangs of a tiger rattlesnake or with the spinal blade of a sea ray, special effector formulation applicator device (mode vehicle) may be utilized to deliver the effector formulation, or a surgical procedure, ideally while using a harmonic scalpel or electro-cautery device, as well as appropriate analgesics, to the specific creature venom toxin injection site should be made such that the effector formulation may be delivered to the specific tissues envenomed by aforementioned specific creature toxin in each case such that the toxin may be efficaciously denatured.
3. Mosquito-borne infection, infectious agent, or illness-causing agent must be treated expeditiously after such person, astutely aware of his/her body and surroundings, notices a mosquito bite and is acutely aware that he/she is in an area endemically prone to these mosquito-borne illnesses and also knows the gravity of contracting such mosquito-borne illness. Typically, the mosquito saliva with the infection, infectious agent, or illness-causing agent is inoculated into the patient's skin cells, by the mosquito, to infect epidermal cells wherein it may replicate before moving to other cells and tissues then to the bloodstream.

There are lots of indications wherein this invention may be utilized to prevent morbidity and mortality in a patient. The following are some examples, not limited to this list, or potential sources of infection, infectious agent, or illness-causing agent wherein the invention may help to prevent, treat, or cure illness in patients. There is an Example-Implementation of Effector formulation as part of a Therapy Regimen Section as part of the examples section that follows.

### Examples

### Bacteria

This invention may be used to prevent, treat, and cure illness caused by an infection, infectious agent, or illness-causing agent through the use of denaturing agents. This invention will work for any bacteria on or in the skin.

Listed are some notable bacterial examples:
1. Aeromonas, with several species causing illness in vertebrate skin, may be associated with boils, carbuncles, feruncles, and pustules.
2. Bacillus anthracis, only the cutaneous form of anthrax serves as an example here, which may present with a blister that may turn into an ulcer and can be deadly.
3. Haemophilus ducreyi, the bacterial infectious agent and cause of Chancroid.
4. Mycobacterium leprae, a bacterial infectious agent and causative agent of Leprosy, transmitted through excessive skin contact with an infected person.
5. Mycobacterium tuberculosis, there are several forms of cutaneous tuberculosis including:
   a. Erythema induratum
   b. Lichen scrofulosorum
   c. Lupus vulgaris
   d. Papulonecrotic tuberculid
   e. Scrofuloderma
   f. Primary inoculation Tuberculosis
6. Mycobacterium ulcerans, a bacterial infectious agent and potential cause of tropical ulcer (jungle rot), is an ulcerative skin lesion.
7. Staphylococcus aureus, has been implicated in skin illnesses including: abscesses, boils, carbuncles, cellulitis, folliculitis, impetigo, pimples, and scalded skin syndrome.
8. Streptococcus pyogenes, a bacterium which produces toxins and causes necrotizing fasciitis.
9. Treponema pallidium, the bacterial infectious agent and causative agent of Chancre.
10. Vibrio vulnificus, may cause necrotizing wound infections. examples of bacterial infection spread by infected fleas:
11. Yersinia pestis is the bacterium responsible for plague which is commonly spread by infected fleas.

examples of bacterial infection spread by infected ticks:
12. Borrelia burgdorferi, is the bacteria, infectious agent, and causative agent of Lyme disease which may be spread by infected ticks.

Lyme disease is reportedly the most common tick-borne illness in the U.S.A.
13. Coxiella burnetti, the bacterial infectious agent and causative agent of Flinders island Spotted fever, Rickettsial Spotted fever, and Q-fever, may be spread by infected ticks.
14. Francisella tularensis, the bacterial infectious agent and causative agent of Tularemia (rabbit fever).

creature venom with toxin
1. Apis mellifera scutellata Lepeletier, Africanized killer Bee, venom includes a mixture of toxic peptides and proteins.
2. Chironex fleckeri, box jellyfish, tentacles contact the skin and nematocysts pierce the skin and inject toxic peptides and proteins.
3. Crotalus tigris, tiger rattlesnake, venom includes a mixture of toxic peptides and proteins.
4. Dasyatis thetidis, a sea ray, is a venomous fish which secretes from the spinal blade venom that includes a mixture of toxic peptides and proteins.
5. Hycleus lugens, blister beetle, venom includes a mixture of toxic peptides and proteins.
6. Latrodectus, widow spider, venom includes a mixture of toxic peptides and proteins.
7. Loxosceles, recluse spider, venom includes a mixture of toxic proteins and peptides which may cause necrosis.
8. Paederus littoralis, rove beetles, venom includes a mixture of toxic peptides and proteins.
9. Paraponera clavata, bullet ant, venom includes a mixture of toxic peptides and proteins.
10. Tityus serrulatus, the Brazilian yellow scorpion, venom which include a toxic mixture of proteins, peptides.
11. Vespula germanica, wasp, venom includes a mixture of toxic peptides and proteins.
12. Vespa mandarinia, Asian giant hornet, venom includes a mixture of toxic peptides and proteins.
13. Vespa orientalis, hornet, venom includes a mixture of toxic peptides and proteins.
14. Vespula squamosa, yellowjacket, venom includes a mixture of toxic peptides and proteins.
   parasite

This invention may be used to prevent, treat, and cure illness caused by an infection, infectious agent, or illness-causing agent through the use of denaturing agents. This invention will work for any parasite on or in the skin.

Listed are some notable parasite examples:
1. Pediculosis, an infestation of lice.
2. Sarcoptes scabies, the mite responsible for the contagious and itchy rash or pimple-like skin condition referred to as scabies.
   insect-borne parasite:
3. Trypanosoma cruzi, the parasitic causative agent of Chagas disease, spread to those who contract the parasite from the bite of the kissing bug, a common mode of transmission. There are millions of people infected in central America, mexico, and south America.
   mosquito-borne parasite:
4. Plasmodium, the parasitic causative agent of malaria, there are hundreds of millions of people infected worldwide.
   plant toxin

This invention may be used to prevent, treat, and cure illness caused by an infection, infectious agent, or illness-causing agent through the use of denaturing agents. This invention will work for any plant toxin on or in the skin. Listed are some notable plant toxin examples:
1. Dendrocnide moroides, the stinging bush, contains toxic peptides and proteins in the leaves and stalks of the plant.
2. Heraceleum mantegazzianum, giant hogweed plant, contains toxic peptides and proteins in the flowers, leaves, roots, seeds, and stems.
3. Urtica dioica, stinging nettle, is a perennial flowering plant that contains hollow stinging trichomes which inject toxic peptides and proteins upon contact.
   virus

This invention may be used to prevent, treat, and cure illness caused by an infection, infectious agent, or illness-causing agent through the use of denaturing agents. This invention will work for any virus on or in the skin.

Listed are some notable virus examples:
mosquito-borne virus:
1. Flavivirus, zika virus, dengue, yellow fever, Japanese encephalitis, west nile viruses all belong to the Flavivirus genus. The aforementioned viruses are spread by mosquitoes. This invention may benefit the patient whom only has contracted the virus very acutely. Typically, the mosquito saliva with the virus is inoculated into the patient's skin cells to infect epidermal cells wherein the virus can replicate and move to other cells and tissues then to the bloodstream.
   skin to skin transmission virus:
1. Herpesviridae, there are several forms of cutaneous herpes including:
   a. Herpes gladitorum, caused by herpes simplex virus 1 or 2, manifestation on the head, torso, or limb of an infected patient's skin.
   b. Herpes simplex 1a viral infection of the central nervous system originating in the skin or mucosa and giving rise to the following manifestations of the systemic disease: disseminated herpes, encephalitis, genital herpes, gingivostomatitis, herpes labials, keratoconjunctivitis, neonatal herpes, pharyngotonsillitis, skin infection.
   c. Herpes simplex 2a viral infection of the central nervous system originating in the skin or mucosa and giving rise to the following manifestations of the systemic disease: disseminated herpes, encephalitis, genital herpes, herpes labials, neonatal herpes, pharyngotonsillitis, skin infection.
   d. Herpetic paronychia (whitlow), caused by herpes simplex virus 1 or 2, and manifestation on the finger or thumb of an infected patient's skin.
      monkey bite transmission virus:
6. Simian Herpes B virus causes herpetic skin lesions near the bite site which may be treated with this invention to defeat this very lethal disease in the patient's skin before it goes to the CNS.
   Animal bite or scratch transmission virus:
7. lyssaviruses are the viral causative agents for Rabies, including rabies virus and Australian bat lyssavirus.

A patient is bitten or scratched by an infected animal whereby the virus infects the patient's epidermal cells wherein the virus replicates before spreading to other organs, tissues, and becoming a systemic disease.

### Other:

skin ulcers caused by bacterial or viral infections may be treated by this invention.

This invention is also not intended to treat bedsores, cancers, chronic wounds due to poor circulation, pressure ulcers,

and also is also not intended to treat venous ulcers.

### Considerations:

This invention allows treatment of a patient with or without definitive diagnosis of illness, infectious agent, or illness-causing agent.

This invention, depending upon treatment therapy regimen employed, may or may not cause scarring or hypo-pigmentation at the specific therapy location of the patient's skin.

This invention may cause scarring or hypo-pigmentation at the specific therapy location of the patient's skin. This may include scarring or hypo-pigmentation to the skin lateral from the specific therapy location of the patient's skin. The likelihood of scarring or hypo-pigmentation to the skin in and around the specific therapy location of the patient's skin increases as: 1. the time the effector formulation is allowed to contact the skin, 2. the strength of any cumulative acid or base component(s) of said effector formulation, and 3. the number of applications of said effector formulation to the specific therapy location of the patient's skin.

### Use of The Invention Technology Case Examples

1. A patient presents with what appears to be a boil or pustule on his leg. The patient does not have insurance or money for labwork including culturing the suspect bacteria to find out definitively which particular genus and species of bacteria is causing the problem, but he wants to be treated and cured right away. The doctor offers to use a local anesthetic, but the patient reports that he has previously had an adverse reaction to the local anesthetic and asks if local anesthetic is required. The doctor says that the treatment regimen at that specific location on the patient would not typically cause too much discomfort. The patient wants to have the treatment as soon as possible. The doctor then begins the therapy regimen whereby an effector formulation of glacial acetic acid is applied to the skin wherein the boil is covered completely with the glacial acetic acid upon administration with a cotton swab at time equals zero, two minutes, and four minutes. when the time equals seven minutes, then the skin including the effector formulation at the specific therapy location is washed off with clean tap water equivalent for five to ten minutes.
2. A patient presents with what appears to be a pus filled sore on his leg. The patient says he had been hunting wild game in a thicket and brushed against a shrub and got a scratch which kind of hurt then he noticed it seemed to blister up for a day, but then it started getting bigger and filling with pus. The patient does not understand why he should get such a sore. The patient just wants the problem on his skin to go away. The doctor is concerned about the look of the sore and thinks it may be necrotizing fasciitis. The doctor tells the patient about the invention and tells the patient that an aggressive treatment may be necessary to prevent an enlarging sore from getting worse and possibly causing amputation or death. The doctor tells the patient about the risk of possible hypopigmentation and scarring at the specific therapy location, but reminds the patient that there is risk of hypopigmentation and scarring risk as well as possible amputation or death even if surgery and debridement of the wound is utilized instead of the invention technology. The doctor offers to use a local anesthetic, and the patient wants the local anesthetic. The doctor says that the treatment regimen at that specific location on the patient may cause some discomfort without the local anesthetic. The patient wants to have the treatment as soon as possible. Before treatment begins, the doctor takes swab samples of the skin illness area and pus to send samples for genus and species identification of bacteria present at the site of suspected infection on the patient's skin and then subsequently begins the therapy regimen whereby an effector formulation of glacial acetic acid is applied to the skin wherein the skin infection area is covered completely with the glacial acetic acid upon administration with a sterile cotton swab at time equals zero and then again every two minutes for sixteen minutes. After 20 minutes has elapsed, the skin including the effector formulation at the specific therapy locations should be washed off with clean tap water equivalent for ten to twenty minutes. Afterwards, it is preferable to dry the area with a blow dryer, but if that is not available it may be patted dry with sterile cotton gauze or dried out carefully with sterile cotton swabs. Air drying may also be a good idea and is simple. After the skin tissue is dry, the specific therapy location on the patient's skin is ideally kept visible such that the specific therapy location may be monitored closely. However, it may be bandaged with sterile dry gauze and tape for securement to keep it clean if the patient has to travel for some reason. the patient may be given systemic antibiotics as part of the therapy regimen. The next day, the patient may have another appointment with the doctor whereby the doctor may evaluate the wound and again take samples of the infection site to send to the lab for culture to determine if the infection site still has active infection. At this point the doctor will help the patient in administering proper systemic antibiotics, keeping the wound clean and helping the patient care for the skin wound until it is completely closed and healed.
3. A patient comes to the doctor after finding two ticks on her leg after taking a walk on Flinders island with her boyfriend and their dog. The patient carefully removes the ticks with tweezers after carefully marking the location of each of the two ticks with an indelible marker on her skin. The patient is really concerned about Flinders Island Spotted fever, because she has heard about the problem from a friend, and she wants to be treated prophylactically right away so that she does not have to worry about Flinders Island Spotted fever, and what it could do to her systemically. The doctor agrees that it would be smart to treat the sites prophylactically although he could not determine whether or not she had contracted or not the responsible bacteria at this point. The doctor explained the invention to the patient, and the patient was happy to start the therapy. So the doctor begins the therapy regimen whereby an effector formulation of glacial acetic acid is applied to the skin wherein the two tick bite locations on the patient's leg are each covered completely with the glacial acetic acid upon administration with a cotton swab at time equals zero, two minutes, four minutes. when the time equals six minutes, then the skin including the effector formulation at the two specific therapy locations should be washed off with clean tap water equivalent for five to ten minutes.
4. A woman goes snorkeling with her friend who happens to be a doctor that is knowledgeable about the invention and has brought her treatment kit onto the boat for the snorkeling adventure just in case something happens. After snorkeling for a while, the woman encounters a box jellyfish wherein the box jellyfish tentacles touch the woman's arm and produce stings on her arm. The woman's arm begins to hurt, so the woman becomes the patient, and signals to her friend the doctor to return to the boat. The doctor carefully removes some remaining nematocysts from the patient's skin with a credit card and subsequently with tweezers also then blots the arm dry with some clean paper towels then marks the skin areas where the stinging sensation occurs as specific therapy locations with an indelible marker on the patient's skin. The patient is really concerned about the danger of box jellyfish venom, and she wants to be treated prophylactically right away so that she will not have to worry about any bad effects from the toxin and what it can do to her systemically. The doctor agrees that she also believes it is smart to treat the sites prophylactically because of the risks associated with the toxin. The doctor explains the invention to the patient, and the patient is happy to start the therapy. So begins the therapy regimen whereby an effector formulation of glacial acetic acid is applied to the skin wherein the stings have occurred and the locations including those wherein the nematocysts are removed from the patient's arm. The doctor covers completely each specific therapy location with the glacial acetic acid upon administration with a cotton swab at time equals zero, two minutes, four minutes. Then when six minutes have elapsed, then the skin including the effector formulation at the specific therapy locations should be washed off with clean tap water equivalent for five to ten minutes.
5. On her day off, a doctor is watching her boyfriend clean his backyard up and then she decides to help him and thus moves a pile of tree limbs to the street. As she is returning to the backyard, the doctor notices a stinging sensation on her arm. The doctor looks at her forearm and notices two small puncture marks, so the doctor believes she may have been bitten by a brown recluse spider because they are plentiful in the area. The doctor had been bitten by a brown recluse spider previously in the same backyard, and it had caused a necrotizing wound that took a long time to heal. She is knowledgeable about the invention, so she goes to her clinic where they conveniently have the new invention technology treatment kit on hand. She begins the therapy regimen whereby an effector formulation of glacial acetic acid is applied to the skin wherein the bite has occurred and at the specific location of the patient's arm. The doctor covers completely the specific therapy location, including the two small puncture wounds, with the glacial acetic acid upon administration with a cotton swab at time equals zero, then every two minutes for four minutes, then after eight minutes the skin including the effector formulation at the specific therapy location is washed off with clean tap water equivalent for five to ten minutes.
6. A man in Washington State, U.S.A., presents after suspected contact with a stinging nettle plant on his arms in a state park. The patient reports that he has never experienced pain like this before, and a short sleeve shirt and straying off the beaten path may have led to the exposure. The E.M.S personnel present at the park explain the invention to the patient. There is no visible swelling on the arms yet, so the patient shows the E.M.S. technician where the arms hurt and the E.M.S. technician removes any noticeable thorny plant material from the patient's skin and then marks lines on the patient's arms to help in application of the effector solution. An effector formulation of glacial acetic acid is applied to the skin on the forearms where the patient says the discomfort or stinging is emanating from wherein the E.M.S. technician covers completely the specific therapy location with the glacial acetic acid upon administration with a cotton gauze pad at time equals zero, and process is repeated two additional times after two minutes elapsed each time with the effector formulation of glacial acetic acid and then covers the exterior skin of the arms thoroughly with the effector formulation of glacial acetic acid and let stand for 4 minutes, then the specific therapy location is washed off with clean tap water equivalent for five to ten minutes.
7. A patient presents with a suspected herpes whitlow lesion on the finger. The patient reports that she has never had such a lesion before, and she is a dental hygienist that was cleaning a patient's teeth when one of her dental instruments pierced her skin after puncturing her latex glove which may have led to the exposure. The doctor explains the invention to the patient. The lesion is visible near the nail toward the tip of the finger, so an effector formulation of glacial acetic acid is applied to the lesion itself wherein the doctor covers completely the specific therapy location with the glacial acetic acid upon administration with a cotton swab at time equals zero, and applying adequately firm but gentile force to the lesion for one minute. Then the doctor gently wets the rest of the finger near the lesion with the effector formulation of glacial acetic acid and then covers the exterior skin of the finger with the effector formulation of glacial acetic acid and let stand for five minutes. The doctor then flushes the lesion and finger such that the specific therapy location is washed off with clean tap water equivalent for ten minutes to twenty minutes.
8. A patient presents with suspected Simian Herpes B virus because a herpetic skin lesion appeared on the back of his hand near the bite site as he works in a research lab that has a study group of monkeys. The patient reports that he has never seen such a lesion before, and told the doctor he was bitten while feeding a monkey in the lab, his workplace, and believes the bite may have led to the exposure which caused this lesion. The doctor explains the invention to the patient. Local and systemic anesthesia is offered to the patient for this procedure. Anesthesia is administered. The lesion is visible on the back of the hand, so an effector formulation of glacial acetic acid is applied to herpetic lesion itself wherein the doctor covers completely the specific therapy location with the glacial acetic acid upon administration with a cotton swab at time equals zero, and applies adequately firm but gentile force to the lesion for one minute. Then the doctor covers the entire hand and wrist including any bite marks or scratches with the effector formulation of glacial acetic acid, and let stand for 10 minutes, then the specific therapy location is washed off with clean tap water equivalent for ten to fifteen minutes.
9. A patient presents with suspected lyssavirus infection, and the parents of the patient are very concerned that their child may have contracted rabies after being bitten on the arms and torso by a strange-acting and vicious-acting dog yesterday. The patient's parents report that she has never seen the dog before and the patient was attacked while walking to visit friends. The parents understand that rabies can be fatal and they don't want to wait to find out if it will become a systemic disease and cannot bear the thought of losing their child to a vicious dog or the fatal disease it may have infected her with. The virus infects patient's epidermal cells wherein the incubation period begins which may be as short as 4 days. The parents do not have the money for the immunoglobulin treatments that may be several thousand dollars per patient treated post-exposure to the rabies virus, so the doctor explains the invention to the patient, as a treatment option for the patient. The patient's parents want the invention treatment to be administered to their child. Local and systemic anesthesia is offered to the patient for this procedure. Anesthesia is administered. The bite marks are visible on both of her arms and one bite mark on her side near her waist, so an effector formulation of glacial acetic acid is applied to all of the bite marks as well as the area around the bite marks wherein the doctor covers completely the specific therapy location with the glacial acetic acid upon administration with a cotton swab at time equals zero, and applying adequately firm but gentile force to the bite marks in each place where the bite penetrated or scratched the skin. Then the doctor treats the bite mark area of the waist with the effector formulation of glacial acetic acid such that all of the bite marks, any scratched skin, and surrounding skin tissue of the patient has been thoroughly treated, and then repeats this entire procedure every two minutes for a total of three applications. Let stand for six additional minutes, then the specific therapy location is washed off with clean tap water equivalent for ten to fifteen minutes.
10. A patient presents with a suspected flea or mosquito bite on his ankle. The patient is doing missionary work in Africa and has been methodical about using mosquito/bug repellent daily because he is working in an area with outbreaks of chickungunya fever, dengue fever, yellow fever, as well as plague. He is using mosquito nets at night, and applies bug repellent a few times a day. He has a fresh bite on the ankle that he believes happened after he finished his shower and before he put the bug repellant on, so he goes to the village hospital. The doctor on-duty looks at the suspected bite and tells the patient about the invention and the treatment. The patient does not care which particular genus and species of bacteria or virus may cause a problem, but he wants to be treated and prevent any infection or illness from occurring right away. The doctor says that the treatment regimen at that specific location on the patient would not typically cause much discomfort. The patient wants to have the treatment as soon as possible. The doctor then begins the therapy regimen whereby an effector formulation of glacial acetic acid is applied to the skin wherein the insect bite location is covered completely with the glacial acetic acid upon administration with a cotton swab at time equals zero, two minutes, and four minutes. when the time equals seven minutes, then the skin including the effector formulation at the specific therapy location is washed off with clean tap water equivalent for five to ten minutes.

I claim:

1. A method for preventing, treating, or curing a patient's illness, comprising the step of:
administering the formulation, effector formulation, treatment, or treatment regimen to a patient whom may be exposed to or has been exposed to an infectious agent or illness-causing agent, wherein the treatment regimen may be applied as a mode by any manner of mode vehicle to a specific therapy location of the patient's body such that the effector formulation treatment denatures protein(s), ribonucleic acid(s), or any component(s) (of a patient's cell or cells, cell or cells of the infection (if present), infectious agent (if present), or illness-causing agent (if present), and infection (if present), infectious agent (if present), or illness-causing agent (if present), any or some or all of the non-cellular matter of the infection (if present), infectious agent (if present), or illness-causing agent (if present) wherein the infection (if present), infectious agent (if present), or illness-causing agent (if present) is not comprised of a cell or cells) that an infection (if present), infectious agent (if present), or illness-causing agent (if present) needs or requires to replicate or to function productively, that a cell needs to replicate or to function productively, or that cells need to replicate or to function productively such that the infection (if present), infectious agent (if present), or illness-causing agent (if present) will not be able to survive, replicate, or further the disease process.

2. The method according to claim 1, wherein the patient's illness refers to the presence of any one or more of the following: infection (if present), infectious agent (if present), or illness-causing agent (if present), in the body of any vertebrate patient (in-vivo).

3. The method according to claim 1, wherein the illness is caused by an infection (if present), infectious agent (if present), or illness-causing agent (if present) such that the source of the illness may be any bacteria, fungus, parasite, toxin, venom, or virus with the specific exception of the viruses that cause warts (papillomaviruses).

4. The method according to claim 1, wherein the formulation is one or more ingredient(s) or any combination of ingredients such that the resulting formulation may be applied to the patient in a specific therapy location, one or more specific therapy location(s), all over the patient's body, or all over the patient's body with the specific exclusion of specific bodily locations, to serve some function as a treatment and as part of a treatment regimen for the patient.

5. The method according to claim 1, wherein the effector formulation is one or more ingredient(s) or any combination of ingredients such that the resulting effector formulation may or may not have an extreme ph (very low or very high, indicating a strong acid or strong base) and may or may not contain non-physiological concentrations of salt(s), organic solvent(s), urea, solution(s), and/or other chemical agent(s), and/or any further ingredient(s) such that the resulting effector formulation may denature, in the specific therapy location beginning upon and continuing for some time after treatment with the effector formulation, the infection (if present), infectious agent (if present), or illness-causing agent (if present) including its proteins and nucleic acids as well as the patient's proteins and nucleic acids within the specific therapy location of the patient's body thereby potentially changing the infection's protein, infectious agent's protein, or illness-causing agent's protein as well as the patient's protein whereby the quaternary structure, tertiary structure, and secondary structure which before the denaturing treatment had been in its native state and as such after the denaturing effects of the effector treatment there is change to some or all of the aforementioned protein in the specific therapy location of the patient's body which may or may not cause some or all of the protein to do any one or more or all of the following: lose their shape, not fit properly, dissociate from sub-units, not function properly biologically, and the denaturing effect of the effector formulation as part of the treatment regimen also may or may not result in unfolding of polypeptides, disruption of intramolecular bonds which may or may not include hydrophobic, electrostatic, and van der waals interactions, disulfide bridges, non-covalent dipole-dipole interactions, and potentially disrupt the alpha-helices and beta-pleated sheets such that they may or may not also lose their respective native configurations and the denaturation effects upon these proteins may or may not be reversible and likewise the nucleic acids present in the same patient's body in the same specific therapy location as well as the infection's nucleic acids, infectious agent's nucleic acids, or illness-causing agent's nucleic acids within the specific therapy location which may or may not function properly biologically because of any one or more or all of the following: losing their shape, potential unfolding of helices, breaking of hydrogen bonds between watson and crick base pairs, such that the cumulative effect of the localized denaturation upon the infection (if present), infectious agent (if present), or illness-causing agent (if present) as well as the patient's own cells and including those patient proteins and nucleic acids within the patient's cells in the specific therapy location of the patient's body will be such that the infection (if present), infectious agent (if present), or illness-causing agent (if present) will not be able to survive, replicate, or further the disease process.

6. The method according to claim 1, wherein a treatment may comprise either a formulation or an effector formulation delivered to the specific therapy location of the patient's body for a specific period of time.

7. The method according to claim 1, wherein the treatment regimen may comprise any number of formulation(s) and/or effector formulation(s) delivered to the specific therapy location of the patient's body each for a prescribed and specific period of time and in a prescribed order which may or may not be followed by one or more additional treatments which may or may not serve as neutralizing step(s) and/or washing step(s) such that the effector formulation(s) is(are) neutralized, neutralized and washed from the patient's body, or directly washed from the patient's body.

8. The method according to claim 1, wherein applied as a mode refers specifically to the embodiment of the product resulting from the any specific formulation or any specific effector formulation: aerosol(s), balm(s), bead(s), bolus(es), cream(s), elixir(s), gel(s), foam(s), liquid(s), lotion(s), nanoparticle(s), nanosuspension(s), ointment(s), particle(s), paste(s), potion(s), powder(s), product(s), salve(s), spray(s), solid(s), solution(s), substance(s), suspension(s), wax(es), as well as any other embodyment(s).

9. The method according to claim 1, wherein applied by any manner of mode vehicle means for the purpose of delivering onto, on, in, upon, into, or around such formulation mode or effector formulation mode onto, on, in, upon, into, or around the specific area of the patient's body to be treated by: applying, bathing, blotting, dabbing, dipping, dripping, dropping, injecting, injecting by hypodermic needle or other injecting apparatus, irrigating, pumping, pouring, shooting, spraying, toweling, wiping, and the like, and some formulation modes or effector formulation modes may or may not be sprayed from a pressurized container or sprayed from pressurized containers, and the pressure for the pressurized container(s) may or may not be supplied by an external means such as squeezing the container or through the use of a mechanical or electric pump(s), or with the use of propellant(s).

10. The method according to claim 1, wherein the treatment regimen may or may not contain an analgesic in one or more of the treatment(s) with or without other ingredients as part of a formulation or effector formulation or be applied to the patient by itself, and in some embodiments systemic analgesia may or may not also be utilized.

11. The method according to claim 1, wherein the treatment regimen may or may not contain an antibacterial medication in one or more of the treatment(s) with or without other ingredients as part of a formulation or effector formulation or be applied to the patient by itself, and in some embodiments systemic antibacterial medication may or may not also be utilized.

12. The method according to claim 1, wherein the treatment regimen may or may not contain an antifungal medication in one or more of the treatment(s) with or without other ingredients as part of a formulation or effector formulation or be applied to the patient by itself, and in some embodiments systemic antifungal medication may or may not also be utilized.

13. The method according to claim 1, wherein the treatment regimen may or may not contain an antiviral medication in one or more of the treatment(s) with or without other ingredients as part of a formulation or effector formulation or be applied to the patient by itself, and in some embodiments systemic antiviral medication may or may not also be utilized.

14. The method according to claim 1, wherein the specific therapy location of the patient's body refers to any specific part, which may be described or defined, of the patient's body, and in a precise location, which refers to the specific location of the patient's body that you can touch, either before or after surgery or any procedure, with your finger, an instrument, tool, or any other device, wherein the treatment regimen should be directed and applied with the most suitable mode and mode vehicle to the specific therapy location, which may include as directed, one or more specific therapy location(s), all over the patient's body, or all over the patient's body with the specific exclusion(s) of specific bodily location(s).

14. The method according to claim 1, wherein the treatment regimen may comprise any aforementioned ingredients or unmentioned ingredients regarding the specific formulation or effector formulation or composition, except for the specific formulation or specific effector formulation or any part of a treatment regimen specifically comprising: about 9 wt. % of lactic acid, about 1.0 wt. % decylene glycol, about 18 wt. % urea, about 3 wt. % NaOH, about 0.05-0.1 wt. % of a hydrolate selected from the group consisting of mint hydrolate, oregano hydrolate, and thymus hydrolate; and an amount of propylene glycol sufficient to bring the composition to 100 wt %.

15. The method according to claim 1, wherein the treatment regimen may comprise any aforementioned ingredients or unmentioned ingredients regarding the specific formulation or effector formulation or composition, except for the specific formulation or specific effector formulation or any part of a treatment regimen specifically comprising only the following ingredient(s): domestic table vinegar (comprised of about 5 wt. % of acetic acid to 10 wt. % acetic acid; and an amount of water sufficient to bring the composition to 100 wt %).

16. The method according to claim 1, wherein the treatment regimen may comprise any aforementioned ingredients or unmentioned ingredients regarding the specific formulation or effector formulation or composition, except for the specific formulation or specific effector formulation or any part of a treatment regimen specifically comprising only the following ingredient(s): domestic household bleach (comprised of about 3 wt. % of Sodium Hypochlorite to 6 wt. % Sodium Hypochlorite; and an amount of water sufficient to bring the composition to 100 wt %).

17. The method according to claim 1, wherein the treatment regimen may comprise any aforementioned ingredients or unmentioned ingredients regarding the specific formulation or effector formulation or composition, including an effector formulation comprised of only one ingredient, an acid, for example: glacial acetic acid, comprising the step of using glacial acetic acid as part of the effector formulation either with or without any other ingredients and solvents either organic or inorganic, and at any concentration in any treatment regimen according to claim 1 for the purpose of denaturing protein(s), ribonucleic acid(s), or any component(s) (of a patient's cell(s), cell or cells of the infection (if present), infectious agent (if present), or illness-causing agent (if present), any or some or all of the non-cellular matter of the infection (if present), infectious agent (if present), or illness-causing agent (if present) wherein the infection (if present), infectious agent (if present), or illness-causing agent (if present) is not comprised of a cell or cells).

18. The method according to claim 1, wherein the treatment regimen may comprise any aforementioned ingredients or unmentioned ingredients regarding the specific formulation or effector formulation or composition, including an effector formulation comprised of only one ingredient, a base, for example: potassium hydroxide, comprising the step of using potassium hydroxide or any other base, as part of the effector formulation either with or without any other ingredients and solvents either organic or inorganic, and at any concentration in any treatment regimen according to claim 1 for the purpose of denaturing protein(s), ribonucleic acid(s), or any component(s) (of a patient's cell(s), cell or cells of the infection (if present), infectious agent (if present), or illness-causing agent (if present), any or some or all of the non-cellular matter of the infection (if present), infectious agent (if present), or illness-causing agent (if present) wherein the infection (if present), infectious agent (if present), or illness-causing agent (if present) is not comprised of a cell or cells).
